(19) United States
(12) Patent Application Publication
(10) Pub. No.: US 2024/0209393 A1
(43) Pub. Date: Jun. 27, 2024

(54) SYNTHETIC AAV VECTORS FOR REPEATED DELIVERY OF THERAPEUTIC GENES

(71) Applicant: University of Florida Research Foundation, Incorporated, Gainesville, FL (US)

(72) Inventors: Arun Srivastava, Gainesville, FL (US); Keyun Qing, Gainesville, FL (US); Jakob Shoti, Gainesville, FL (US)

(73) Assignee: University of Florida Research Foundation, Incorporated, Gainesville, FL (US)

(21) Appl. No.: 18/288,301

(22) PCT Filed: Apr. 25, 2022
(86) PCT No.: PCT/US2022/026150

§ 371 Date: Oct. 25, 2023

Related U.S. Application Data
(60) Provisional application No. 63/180,056, filed on Apr. 26, 2021.

(51) Int. Cl. C12N 15/86; C07K 14/755; C12N 9/64

(52) U.S. Cl. CPC C12N 15/86; C07K 14/755; C12N 9/644; C12Y 304/21022; C12N 2750/14143; C12N 2830/008

(57) ABSTRACT

The present invention provides optimized no-end adeno-associated virus (NE-AAV) DNA genomes comprising recombinant AAV inverted terminal repeats (ITRs), compositions comprising the same, and methods of use thereof to treat certain diseases or disorders. Such genomes and compositions may be useful in the delivery of therapeutic genes to subjects in need thereof, for example in the treatment of certain diseases or disorders known to be associated with the liver, such as hemophilia. The methods of the disclosure may be useful in the site-specific integration of NE-DNA and treatment of hemophilia in humans, including children.

### RELATED APPLICATIONS

This application claims the benefit under 35 U.S.C. § 119(e) of the filing date of U.S. Provisional Application Ser. No. 63/180,056, filed Apr. 26, 2021, entitled “SYNTHETIC AAV VECTORS FOR REPEATED DELIVERY OF THERAPEUTIC GENES”, the entire content of which is incorporated herein by reference in its entirety.

### SEQUENCE LISTING

In accordance with 37 C.F.R. 1.52(e)(5), the present specification makes reference to a Sequence Listing (submitted electronically as a .txt file named “U119670086WO00-SEQ-KSB”). The .txt file was generated on Apr. 22, 2022 and is 3,443 bytes in size. The Sequence Listing is herein incorporated by reference in its entirety.

### BACKGROUND

Recombinant AAV vectors have been used for the gene therapy of liver diseases in several clinical trials in adult patients. However, none of these clinical trials have included children with liver diseases, because child patients face specific challenges which render traditional liver-directed AAV gene therapies ineffective. For example, the human liver continues growing and dividing until age 10-12, and with every cell division AAV vector genomes are diluted out due to their episomal nature. Additionally, necessary repeat dosing, even with an ideal AAV vector, is challenging in children because of pre-existing antibodies to AAV vectors following the first administration.

### SUMMARY

A solution is therefore needed to safely and effectively deliver gene therapies via recombinant AAV vectors to children having liver diseases, such as hemophilia.

The present invention provides no-end adeno-associated virus (NE-AAV) DNA genomes comprising recombinant AAV inverted terminal repeats (ITRs), compositions comprising the same, and methods of use thereof to treat certain diseases or disorders. The NE-AAV genomes (e.g., double-stranded, linear DNA with 7 unpaired nucleotides at each of the 5′ and 3′ ends) of the present disclosure were developed based upon a previously-described hybrid genome that consists of a gene cassette covalently flanked by AAV ITRs, which were each 165 nucleotides in length, with no open ends (see Nahrcini, et al., Gene (1992), 119: 265-272).

In a surprising advance over the previously-described hybrid genomes, recombinant AAV ITRs are provided herein which are truncated and/or modified relative to full-length AAV ITRs of the same serotype. In some embodiments, such truncations and/or modifications may increase the stability of the NE-AAV genome (e.g., increase the thermodynamic stability of the no-end rAAV genome) and/or increase ligation efficiency, and thereby increase transgene expression (e.g., increase the level and/or stability of transgene expression) and/or increase the longevity of transgene expression (e.g., increase the stability of transgene expression).

In some embodiments, such truncations may be made to one AAV ITR, at either end of a NE-AAV. In other embodiments, such truncations may be made to both AAV ITRs, at both ends of a NE-AAV. In embodiments including truncations made to both AAV ITRs (at both ends of a NE-AAV), the truncations may be the same, or may be different (e.g., the truncated AAV ITR located at one end of a NE-AAV may be of a different length than the truncated AAV ITR located at the other end of the NE-AAV; or the AAV ITRs may be of the same length, but comprise different nucleotide deletions).

Aspects of the invention relate to the use of the disclosed NE-AAV DNA genomes to express a transgene following delivery into mammalian cells. Administration of the NE-AAV genomes of the disclosure may thus in some embodiments be useful in the treatment of certain diseases or disorders—for example certain diseases or disorders known to be associated with the liver, such as hemophilia. Administration of the NE-AAV DNA genomes comprising transgenes known to be useful in the treatment of hemophilia to human children subjects in need thereof is specifically contemplated herein.

Aspects of the invention include a no end-rAAV genome comprising a heterologous gene flanked by recombinant AAV ITRs. In some embodiments, at least one of the recombinant AAV ITRs is a truncated AAV ITR having between about 35 and 95 nucleotides, wherein the truncated AAV ITR is truncated relative to a full-length AAV ITR of the same serotype. In some embodiments, the truncated AAV ITR has about 55-95 nucleotides. In other embodiments, the truncated AAV ITR has about 75-95 nucleotides. In some embodiments, the truncated AAV ITR has about 35, about 55, about 75, or about 95 nucleotides. In some embodiments, the truncated AAV ITR has about 75 nucleotides. In some embodiments, the truncated AAV ITR has about 35 nucleotides. In some embodiments, the truncated AAV ITR has about 55 nucleotides. In some embodiments, the truncated AAV ITR has about 95 nucleotides.

In some embodiments, the no-end rAAV genome does not comprise a nucleotide sequence capable of encoding AAV capsid protein(s).

In some embodiments, the truncated AAV ITR further comprises one or more nucleic acid mutations, relative to a full-length AAV ITR of the same serotype. In some embodiments, the nucleic acid mutations are stabilizing mutations and/or result in more efficient ligation than a truncated AAV ITR which does not comprise said mutation(s).

In some embodiments, the truncated AAV ITR is truncated relative to a full-length ITR of a wild-type AAV serotype, including, but not limited to, serotypes 1, 2, 3, 4, 5, or 6. In some embodiments, the truncated AAV ITR is truncated relative to any one of SEQ ID NOs: 1-6. In some embodiments, the truncated AAV ITR is truncated relative to a full-length ITR of AAV serotype 2. In some embodiments, the truncated AAV ITR is truncated relative to SEQ ID NO: 2.

In some embodiments, the truncated AAV ITR comprises a nucleotide sequence having at least 90%, at least 95%, at least 98%, or at least 99% identity to the nucleotide sequence of any one of SEQ ID NOs: 7-11. In some embodiments, the truncated AAV ITR comprises the nucleotide sequence of any one of SEQ ID NOs: 7-11. In some embodiments, the truncated AAV ITR comprises the nucleotide sequence of SEQ ID NO: 9. In some embodiments, the truncated AAV ITR comprises the nucleotide sequence of SEQ ID NO: 7. In some embodiments, the truncated AAV ITR comprises the nucleotide sequence of SEQ ID NO: 8. In some embodiments, the truncated AAV ITR comprises the nucleotide sequence of SEQ ID NO: 10. In some embodiments, the truncated AAV ITR comprises the nucleotide sequence of SEQ ID NO: 11.

In some embodiments, the heterologous gene is a gene associated with one or more of the following diseases or disorders: Alpha 1-Antitrypsin Deficiency, Phenylketonuria, Wilson Disease, Acute Intermittent Porphyria, Familial Hypercholesterolemia, Crigler-Najjar Syndrome, ATTR Amyloidosis, Methylmalonic Acidemia, a Mucopolysaccharidosis, Glycogen Storage Disease Type Ia, Ornithine Transcarbamylase Deficiency, liver fibrosis, Hemophilia A, Hemophilia B, and Hemophilia C. In some embodiments, the heterologous gene is a gene associated with one or more of the following diseases or disorders: liver fibrosis, Hemophilia A. Hemophilia B, and Hemophilia C.

In some embodiments, the heterologous gene is or comprises a: Serpin Family A Member 1 (SERPINA1) gene, gene encoding Phenylalanine Hydroxylase (PAH), ATPase Copper Transporting Beta (ATPB7) gene, Hydroxymethylbilane Synthase (HMBS) gene, low density lipoprotein receptor (LDLR) gene, UDP Glucuronosyltransferase Family 1 Member A Complex Locus (UGTIA) gene, transthyretin (TTR) gene, methylmalonyl Coenzyme A mutase (MMUT) gene, Mucopolysaccharidosis Type I (MPS I) gene, Mucopolysaccharidosis Type II (MPS II) gene, Mucopolysaccharidosis Type IIIA (MPS IIIA) gene, Mucopolysaccharidosis Type IIIB (MPS IIIB) gene, Mucopolysaccharidosis Type IIIC (MPS IIIC) gene, Mucopolysaccharidosis Type IIID (MPS IIID) gene, Mucopolysaccharidosis Type IVA (MPS IVA) gene, Mucopolysaccharidosis Type IVB (MPS IVB) gene, Mucopolysaccharidosis Type V (MPS V) gene, Mucopolysaccharidosis Type VI (MPS VI) gene, Mucopolysaccharidosis Type VII (MPS VII) gene, Mucopolysaccharidosis Type IX (MPS IX) gene, glucose-6-phosphatase catalytic-subunit (G6PC) gene, Ornithine Transcarbamylase (OTC) gene, gene encoding human serum albumin, gene encoding clotting Factor VIII (F.VIII), or gene encoding clotting Factor IX (F.IX).

In some embodiments, the heterologous gene encodes: α-1 anti-trypsin protein, phenylalanine hydroxylase, ATPase Copper Transporting Beta, porphobilinogen deaminase, Hydroxymethylbilane Synthase, low-density lipoprotein receptor, uridine diphosphoglucuronate glucuronosyltransferase, transthyretin protein, methylmalonyl Coenzyme A mutase, α-L-iduronidase, iduronate sulfatase, heparan N-sulfatase, α-N-acetylglucosaminidase, acetyl-CoA α-glucosaminide acetyltransferase, N-acetylglucosamine 6-sulfatase, N-acetylgalactosamine-6-sulfatase. β-galactosidase, α-L-iduronidase, N-acetylgalactosamine 4-sulfatase, β-glucuronidase, hyaluronidase, glucose-6-phosphatase, ornithine Transcarbamylase, human serum albumin, clotting Factor VIII (F.VIII), or clotting Factor IX (F.IX). In some embodiments, the heterologous gene encodes albumin, Factor VIII (F.VIII), or Factor IX (F.IX).

In some embodiments, a no-end rAAV genome as described herein further comprises a promoter. In some embodiments, the promoter is a liver-specific promoter and/or a liver cancer cell-specific promoter. In some embodiments, the promoter comprises any one of: human liver cell-specific trans-thyretin (TTR) promoter, human α-fetoprotein promoter (AFP), human al-antitrypsin (hAAT) promoter, human F.IX promoter, human F.VIII promoter, and liver promoter 1 (LP1).

In some embodiments, both recombinant AAV ITRs are truncated, relative to a full-length AAV ITR of the same serotype.

Aspects of the invention include a composition comprising a no-end rAAV genome as described herein. In some embodiments, the composition comprises an AAV particle. In some embodiments, the AAV particle is an AAV serotype 1, 2, 3, 4, 5, 6, 7, 8, 9, 10, 11, 12, or 13 particle. In some embodiments, the composition comprises a liposome. In some embodiments, the liposome comprises a galactosylated lipid. In some embodiments, the composition comprises a nanoparticle. In some embodiments, the nanoparticle comprises a lipid nanoparticle. In some embodiments, the composition comprises an exosome. In some embodiments, the exosome comprises an exosome-associated rAAV.

Aspects of the invention include a method of treating a disease or disorder, the method comprising administering a no-end rAAV genome as described herein or a composition comprising a no-end rAAV genome as described herein to a subject in need thereof. In some embodiments, the subject is a human. In some embodiments, the subject is a human child. In some embodiments, the no-end rAAV genome or the composition is administered intramuscularly, intravenously, subcutaneously, intrathecally, intraperitoneally, or by direct injection into an organ or a tissue of the subject. In some embodiments, the disease or disorder is selected from the group consisting of: Alpha 1-Antitrypsin Deficiency, Phenylketonuria, Wilson Disease, Acute Intermittent Porphyria, Familial Hypercholesterolemia, Crigler-Najjar Syndrome, ATTR Amyloidosis, Methylmalonic Acidemia, a Mucopolysaccharidosis, Glycogen Storage Disease Type Ia, Ornithine Transcarbamylase Deficiency, liver fibrosis, Hemophilia A, Hemophilia B, and Hemophilia C. In some embodiments, the disease or disorder is selected from the group consisting of: liver fibrosis, Hemophilia A, Hemophilia B, and Hemophilia C.

In some embodiments, a no-end rAAV genome of the disclosure or a composition comprising a no-end rAAV genome of the disclosure further comprises a nucleotide sequence encoding an AAV Rep protein. In some embodiments, the heterologous gene is integrated into human chromosome 19 (19q13.3).

Aspects of the invention include a recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 9. In some embodiments, the recombinant AAV ITR comprises the nucleotide sequence of SEQ ID NO: 9.

Aspects of the invention include a recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 7. In some embodiments, the recombinant AAV ITR comprises the nucleotide sequence of SEQ ID NO: 7.

Aspects of the invention include a recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 8. In some embodiments, the recombinant AAV ITR comprises the nucleotide sequence of SEQ ID NO: 8.

Aspects of the invention include a recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 10. In some embodiments, the recombinant AAV ITR comprises the nucleotide sequence of SEQ ID NO: 10.

Aspects of the invention include a recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 11. In some embodiments, the recombinant AAV ITR comprises the nucleotide sequence of SEQ ID NO: 11.

### BRIEF DESCRIPTION OF THE DRAWINGS

FIGS. 1A and 1B show schematic structures of the AAV inverted terminal repeat (ITR) and linear and No-End (NE) DNAs of various lengths. FIG. 1A shows a schematic structure of the AAV2 ITR (SEQ ID NO: 2) in its hairpin configuration. Truncated (“TR”) AAV-ITRs of various indicated lengths are also shown: 35 nucleotides (“TR35”; SEQ ID NO: 11); 55 nucleotides (“TR55”; SEQ ID NO: 8); 75 nucleotides (“TR75”; SEQ ID NO: 9); and 95 nucleotides (“TR95”; SEQ ID NO: 10). FIG. 1B shows schematic structures of linear and NE Enhanced Green Fluorescence Protein (EGFP) expression cassettes under the control of the human α-fetoprotein promoter (AFP) flanked by truncated AAV-ITRs of various indicated lengths: 35 nucleotides (TR35); 55 nucleotides (TR55); 75 nucleotides (TR75); and 95 nucleotides (TR95).

FIGS. 2A and 2B show EGFP expression in Huh7 cells with plasmid, linear, and No End (NE) DNAs 3-days post-transfection. FIG. 2A shows EGFP expression quantified as pixel²/visual field×10⁴ for the linear, NE-TR35, NE-TR55, NE-TR75, and NE-TR95 DNAS. FIG. 2B shows EGFP expression for the Linear-AFP-EGFP DNA (top) and the NE-TR75-AFP-EGFP DNA (bottom).

FIGS. 3A and 3B show transgene (EGFP) expression in Huh7 cells with plasmid, linear, and NE-DNAs 28-days post-transfection. FIG. 3A shows EGFP expression with ±1 standard deviation error quantified as pixel²/visual field×10⁴ for the plasmid, linear, NE-TR35, NE-TR55, NE-TR75, and NE-TR95 DNAs. FIG. 3B shows EGFP expression for the Linear-AFP-EGFP DNA (top) and the NE-TR75-AFP-EGFP DNA (bottom).

FIG. 4 shows agarose gel electrophoresis of PCR-amplified fragments from low molecular weight DNAs from transfected cells 35-days post-transfection. Linear, NE-TR75, and plasmid (p) DNA constructs containing a EGFP expression cassette under the control of the human liver cell-specific trans-thyretin (TTR) promoter are shown from left to right, respectively. The expected 1.5 kb DNA fragments and positive [+] and negative [−] controls are also shown.

FIGS. 5A and 5B show the development of synthetic AAV NE-Factor IX [NE-hF.IX] and Factor VIII [NE-hF. VIII] liposome vectors, and NE-hF.IX and -hF.VIII exosome vectors. FIG. 5A shows liposome systems. FIG. 5B shows exosome systems.

FIGS. 6A and 6B show the increased expression of human Factor IX (hF.IX) from NE-TR75 TTRp-hF.IX DNA in a human liver cell line in vitro. FIG. 6A shows western blot analysis of hF.IX expression in human HepG2 cells 7 days, 14 days, and 28 days following mock-transfection (M), transfection with linear-TTRp-hF.IX (L), or transfection with NE-TR75 TTRp-hF.IX (N) DNA using monoclonal anti-hF.IX antibody. FIG. 6B shows quantitation of the data from FIG. 6A for 14 and 28 days post-transfection. NE=No-end; hF.IX=human Factor IX; TTRp=human transthyretin promoter.

### DETAILED DESCRIPTION

The present invention provides no-end adeno-associated virus (NE-AAV) DNA genomes (e.g., double-stranded, linear DNA with 7 unpaired nucleotides at each of the 5′ and 3′ ends) comprising AAV recombinant inverted terminal repeats (ITRs), compositions comprising the same, and methods of use thereof to treat certain diseases or disorders. Recombinant AAV ITRs are provided herein which are truncated and/or modified relative to full-length AAV ITRs of the same serotype.

In some embodiments, such truncations and/or modifications may increase the stability of the no end-rAAV genome (e.g., increase the thermodynamic stability of the no-end rAAV genome) and/or increase ligation efficiency, and thereby increase transgene expression (e.g., increase the level and/or stability of transgene expression) and/or increase the longevity of transgene expression (e.g., increase the stability of transgene expression). In some embodiments, such truncations may be made to one AAV ITR, at either end of a NE-AAV. In other embodiments, such truncations may be made to both AAV ITRs, at both ends of a NE-AAV. In embodiments including truncations made to both AAV ITRs (at both ends of a NE-AAV), the truncations may be the same, or may be different (e.g., the truncated AAV ITR located at one end of a NE-AAV may be of a different length than the truncated AAV ITR located at the other end of the NE-AAV; or the AAV ITRs may be of the same length, but comprise different nucleotide deletions).

Aspects of the invention relate to the use of the disclosed no end-rAAV DNA genomes to express a transgene following delivery into mammalian cells. Administration of the no end-rAAV genomes of the disclosure may thus in some embodiments be useful in the treatment of certain diseases or disorders—for example certain diseases or disorders known to be associated with the liver, such as hemophilia. Administration of the no end-rAAV DNA genomes comprising transgenes known to be useful in the treatment of hemophilia to human children subjects in need thereof is specifically contemplated herein.

### No-End Adeno-Associated Virus DNA (NE-AAV DNA) Constructs

Aspects of the present disclosure relate to no-end rAAV genomes comprising recombinant AAV ITRs flanking heterologous genes, and compositions comprising the same. As used herein, a “no-end” AAV genome refers to a continuous strand of DNA joined at each end by the AAV ITRs, resulting in a continuous double-stranded DNA molecule. No-end AAV genomes are described, for example, in Nahreini, et al. (Gene (1992), 119: 265-72), which is herein incorporated by reference in its entirety. In some embodiments, the no-end rAAV genome does not comprise a nucleotide sequence capable of encoding AAV capsid protein(s). In some embodiments, a no-end rAAV genome of the disclosure further comprises a nucleotide sequence encoding an AAV Rep protein.

As used herein, a “heterologous gene” or a “transgene” refers to a gene or coding sequence of a gene which is not associated with AAV in its natural, wild-type context, and which is positioned between AAV ITRs in the no-end AAV genomes of the disclosure. In some embodiments, a heterologous gene (e.g., transgene) is encoded or is expressed following delivery of the no-end AAV genome(s) into cells.

A nucleic acid vector as disclosed herein in some embodiments comprises one or more regulatory elements. A regulatory element refers to a nucleotide sequence or structural component of a nucleic acid vector which is involved in the regulation of expression of components of the nucleic acid vector (e.g., a gene of interest comprised therein). Regulatory elements include, but are not limited to, promoters, enhancers, silencers, insulators, response elements, initiation sites, termination signals, and ribosome binding sites. In some embodiments, a no-end rAAV genome as described herein further comprises a promoter.

Promoters include constitutive promoters, inducible promoters, tissue-specific promoters, cell type-specific promoters, and synthetic promoters. For example, a nucleic acid vector disclosed herein may include viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter. Inducible promoters or other inducible regulatory elements may also be used to achieve desired expression levels of a gene of interest (e.g., a protein or polypeptide of interest). Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue- and cell-specific promoters or other tissue- or cell-specific regulatory elements are also contemplated herein. In some embodiments, the promoter is a liver-specific promoter and/or a liver cancer cell-specific promoter. In some embodiments, the promoter comprises any one of: human liver cell-specific trans-thyretin (TTR) promoter, human α-fetoprotein promoter (AFP), human al-antitrypsin (hAAT) promoter, human F.IX promoter, human F.VIII promoter, and liver promoter 1 (LP1).

Synthetic promoters are also contemplated herein. A synthetic promoter may comprise, for example, regions of known promoters, regulatory elements, transcription factor binding sites, enhancer elements, repressor elements, and the like.

In some embodiments, a no end-rAAV genome as provided herein comprises a heterologous gene capable of encoding or expressing a product (e.g., a protein or polypeptide product, or an RNA, etc.). In some embodiments, a heterologous gene comprises a nucleotide sequence of a gene of interest. In some embodiments, a heterologous gene encodes a therapeutic or diagnostic protein or polypeptide. In some embodiments, a therapeutic or diagnostic protein or polypeptide is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic agent, an enzyme, a bone morphogenetic protein, a nuclease, guide RNA or other nucleic acid or protein used for gene editing, an Fc-fusion protein, an anticoagulant, or a protein or polypeptide that can be detected using a laboratory test.

In some embodiments, a heterologous gene is a gene associated with one or more of the following diseases or disorders: Alpha 1-Antitrypsin Deficiency, Phenylketonuria, Wilson Disease, Acute Intermittent Porphyria, Familial Hypercholesterolemia, Crigler-Najjar Syndrome, ATTR Amyloidosis, Methylmalonic Acidemia, a Mucopolysaccharidosis, Glycogen Storage Disease Type Ia, Ornithine Transcarbamylase Deficiency, liver fibrosis, Hemophilia A, Hemophilia B, and Hemophilia C. In some embodiments, the heterologous gene is a gene associated with one or more of the following diseases or disorders: liver fibrosis, Hemophilia A, Hemophilia B, and Hemophilia C.

In some embodiments, the heterologous gene is or comprises a: Serpin Family A Member 1 (SERPINA1) gene, gene encoding Phenylalanine Hydroxylase (PAH), ATPase Copper Transporting Beta (ATPB7) gene, Hydroxymethylbilane Synthase (HMBS) gene, low density lipoprotein receptor (LDLR) gene, UDP Glucuronosyltransferase Family 1 Member A Complex Locus (UGTIA) gene, transthyretin (TTR) gene, methylmalonyl Coenzyme A mutase (MMUT) gene, Mucopolysaccharidosis Type I (MPS I) gene, Mucopolysaccharidosis Type II (MPS II) gene, Mucopolysaccharidosis Type IIIA (MPS IIIA) gene, Mucopolysaccharidosis Type IIIB (MPS IIIB) gene, Mucopolysaccharidosis Type IIIC (MPS IIIC) gene, Mucopolysaccharidosis Type IIID (MPS IIID) gene, Mucopolysaccharidosis Type IVA (MPS IVA) gene, Mucopolysaccharidosis Type IVB (MPS IVB) gene, Mucopolysaccharidosis Type V (MPS V) gene, Mucopolysaccharidosis Type VI (MPS VI) gene, Mucopolysaccharidosis Type VII (MPS VII) gene, Mucopolysaccharidosis Type IX (MPS IX) gene, glucose-6-phosphatase catalytic-subunit (G6PC) gene, Ornithine Transcarbamylase (OTC) gene, gene encoding human serum albumin, gene encoding clotting Factor VIII (F.VIII), or gene encoding clotting Factor IX (F.IX).

In some embodiments, the heterologous gene encodes: α-1 anti-trypsin protein, phenylalanine hydroxylase, ATPase Copper Transporting Beta, porphobilinogen deaminase, Hydroxymethylbilane Synthase, low-density lipoprotein receptor, uridine diphosphoglucuronate glucuronosyltransferase, transthyretin protein, methylmalonyl Coenzyme A mutase, α-L-iduronidase, iduronate sulfatase, heparan N-sulfatase, α-N-acetylglucosaminidase, acetyl-CoA α-glucosaminide acetyltransferase, N-acetylglucosamine 6-sulfatase, N-acetylgalactosamine-6-sulfatase, β-galactosidase, α-L-iduronidase, N-acetylgalactosamine 4-sulfatase, β-glucuronidase, hyaluronidase, glucose-6-phosphatase, ornithine Transcarbamylase, human serum albumin, clotting Factor VIII (F.VIII), or clotting Factor IX (F.IX). In some embodiments, the heterologous gene encodes albumin, Factor VIII (F.VIII), or Factor IX (F.IX).

In some embodiments, a product encoded by a heterologous gene disclosed herein is a detectable molecule. A detectable molecule is a molecule that can be visualized (e.g., using a naked eye, under a microscope, or using a light detection device such as a camera). In some embodiments, the detectable molecule is a fluorescent molecule, a bioluminescent molecule, or a molecule that provides color (e.g., β-galactosidase, β-lactamase, β-glucuronidase, or spheroidenone). In some embodiments, the detectable molecule is a fluorescent, bioluminescent or enzymatic protein or functional peptide or polypeptide thereof.

In some embodiments, fluorescent protein is a blue fluorescent protein, a cyan fluorescent protein, a green fluorescent protein, a yellow fluorescent protein, an orange fluorescent protein, a red fluorescent protein, or a functional peptide or polypeptide thereof. A blue fluorescent protein may be azurite, EBFP. EBFP2, mTagBFP, or Y66H. A cyan fluorescent protein may be ECFP, AmCyan1, Cerulean, CyPet, mECFP, Midori-ishi Cyan, mTFP1, or TagCFP. A green fluorescent protein may be AcGFP, Azami Green, EGFP, Emarald, GFP or a mutated form of GFP (e.g., GFP-S65T, mWasabi, Stemmer, Superfolder GFP. TagGFP. TurboGFP, or ZsGreen). A yellow fluorescent protein may be EYFP, mBanana, mCitrine, PhiYFp, TagYFP, Topaz, Venus, YPet, or Zs Yellow1. An orange fluorescent protein may be DsRed, RFP, DsRed2, DsRed-Express, Ds-Red-monomer, Tomato, tdTomato, Kusabira Orange, mKO2, mOrange, mOrange2, mTangerine, TagRFP, or TagRFP-T. A red fluorescent protein may be AQ142, AsRed2, dKeima-Tandem, HcRed1, tHcRed, Jred, mApple, mCherry, mPlum, mRaspberry, mRFP1, mRuby or mStrawberry.

In some embodiments, a detectable molecule is a bioluminescent protein or a functional peptide or polypeptide thereof. Non-limiting examples of bioluminescent proteins are firefly luciferase, click-beetle luciferase, Renilla luciferase, and luciferase from Oplophorus gracilirostris.

In some embodiments, a detectable molecule may be any polypeptide or protein that can be detected using methods known in the art. Non-limiting methods of detection are fluorescence imaging, luminescent imaging, bright field imaging, and imaging facilitated by immunofluorescence or immunohistochemical staining.

Additional features of AAV particles, nucleic acid vectors, and capsid proteins are described in U.S. Pat. No. 10,900,053, the content of which is incorporated herein by reference in its entirety.

### Recombinant Inverted Terminal Repeats (ITRs)

Aspects of the present disclosure relate to no end-rAAV DNA genomes comprising a heterologous gene flanked by recombinant ITRs. In some embodiments, recombinant AAV ITRs are provided herein which are truncated and/or modified relative to full-length AAV ITRs of the same AAV serotype. Such truncations and/or modifications may in some embodiments increase the stability of the no end-rAAV genome (e.g., increase the thermodynamic stability of the no-end rAAV genome) and/or increase ligation efficiency, and thereby increase transgene expression (e.g., increase the level and/or stability of transgene expression) and/or increase the longevity of transgene expression (e.g., increase the stability of transgene expression).

In some embodiments, such truncations may be made to one ITR, at either end of a NE-AAV. In other embodiments, such truncations may be made to both ITRs, at both ends of a NE-AAV. In embodiments including truncations made to both ITRs (at both ends of a NE-AAV), the truncations may be the same, or may be different (e.g., the truncated AAV ITR located at one end of a NE-AAV may be of a different length than the truncated AAV ITR located at the other end of the NE-AAV; or the AAV ITRs may be of the same length, but comprise different nucleotide deletions).

As will be understood, each no end-rAAV genome comprises ITRs at both ends (e.g., the 5′, or left end, and the 3′, or right end) of the continuous DNA strand. Full-length (e.g., wild-type) AAV ITRs typically comprise around 145 bases each (see Table 1), and have been shown to be required for efficient multiplication of the AAV genome. ITR sequences have the ability to form a hairpin structure (e.g., a stem/loop structure), and contribute to both integration of the AAV DNA into the host cell genome and rescue from it, as well as efficient encapsidation of the AAV DNA combined with generation of a fully-assembled, deoxyribonuclease-resistant AAV particles.

As used herein, a “recombinant” AAV ITR refers to an AAV ITR which is modified, relative to a full-length AAV ITR of the same AAV serotype. Such modification(s) may comprise the addition, substitution, and/or deletion of one or more nucleotides of the AAV ITR nucleotide sequence.

In some embodiments, a recombinant AAV ITR comprises an addition of one or more nucleotides to the AAV ITR nucleotide sequence, relative to a full-length AAV ITR of the same AAV serotype. In some embodiments, the addition of one or more nucleotides to the AAV ITR nucleotide sequence increases ligation efficiency. In some embodiments, the addition of one or more nucleotides to the AAV ITR nucleotide sequence increases transgene expression. In some embodiments, the addition of one or more nucleotides to the AAV ITR nucleotide sequence increases the longevity of transgene expression.

In some embodiments, a recombinant AAV ITR comprises a mutation of one or more nucleotides of the AAV ITR nucleotide sequence, relative to a full-length AAV ITR of the same AAV serotype. In some embodiments, the mutation comprises a substitution of one or more nucleotides of the AAV ITR nucleotide sequence, relative to a full-length AAV ITR of the same AAV serotype. In some embodiments, the substitutions of one or more nucleotides of the AAV ITR nucleotide sequence increase the stability of the NE-AAV genome (e.g., increase the thermodynamic stability of the no-end rAAV genome). In some embodiments, the substitution of one or more nucleotides of the AAV ITR nucleotide sequence increases ligation efficiency. In some embodiments, the substitution of one or more nucleotides of the AAV ITR nucleotide sequence increases transgene expression (e.g., increases the level and/or stability of transgene expression). In some embodiments, the substitution of one or more nucleotides of the AAV ITR nucleotide sequence increases the longevity of transgene expression (e.g., increases the stability of transgene expression).

In some embodiments, such mutation comprises the substitution of a terminal thymine (T) nucleic acid for a guanine (G) nucleic acid. In some embodiments, such mutation comprises the substitution of a terminal guanine (G) nucleic acid for a thymine (T) nucleic acid. In some embodiments, such mutation comprises the substitution of a terminal adenine (A) nucleic acid for a cytosine (C) nucleic acid. In some embodiments, such mutation comprises the substitution of a terminal cytosine (C) nucleic acid for an adenine (A) nucleic acid. In some embodiments, such mutation comprises the substitution of a terminal thymine (T) nucleic acid for a cytosine (C) nucleic acid. In some embodiments, such mutation comprises the substitution of a terminal cytosine (C) for a thymine (T) nucleic acid. In some embodiments, such mutation comprises the substitution of a terminal thymine (T) nucleic acid for an adenine (A) nucleic acid. In some embodiments, such mutation comprises the substitution of a terminal adenine (A) for a thymine (T) nucleic acid. In some embodiments, such mutation comprises the substitution of a terminal guanine (G) nucleic acid for a cytosine (C) nucleic acid. In some embodiments, such mutation comprises the substitution of a terminal cytosine (C) nucleic acid for a guanine (G) nucleic acid. In some embodiments, such mutation comprises the substitution of a terminal guanine (G) nucleic acid for an adenine (A) nucleic acid. In some embodiments, such mutation comprises the substitution of a terminal adenine (A) nucleic acid for a guanine (G) nucleic acid.

In some embodiments, a recombinant AAV ITR comprises a truncated AAV ITR. As used herein, a “truncated” AAV ITR is an AAV ITR which comprises a deletion of one or more nucleotides of the AAV ITR nucleotide sequence, relative to a full-length AAV ITR of the same AAV serotype. In some embodiments, one recombinant AAV ITR (e.g., the 5′-AAV ITR or the 3′-AAV ITR) is truncated, relative to a full-length AAV ITR of the same serotype. In some embodiments, both recombinant AAV ITRs (e.g., the 5′-AAV ITR and the 3′-AAV ITR) are truncated, relative to a full-length AAV ITR of the same serotype. In some embodiments, the deletion of one or more nucleotides of the AAV ITR nucleotide sequence increases ligation efficiency. In some embodiments, the deletion of one or more nucleotides of the AAV ITR nucleotide sequence increases transgene expression (e.g., increases the level and/or stability of transgene expression). In some embodiments, the deletion of one or more nucleotides of the AAV ITR nucleotide sequence increases the longevity of transgene expression (e.g., increases the stability of transgene expression).

Accordingly, in some embodiments at least one of the recombinant AAV ITRs is a truncated AAV ITR having (e.g., retaining; comprising) between about 35 and 95 AAV ITR nucleotides, wherein the truncated AAV ITR is truncated relative to a full-length AAV ITR of the same serotype. In some embodiments, the truncated AAV ITR has about 55-95 nucleotides. In some embodiments, the truncated AAV ITR has about 30-40, about 35-45, about 40-50, about 45-55, about 50-60, about 55-65, about 60-70, about 65-75, about 70-80, about 75-85, about 80-90, about 85-95, about 90-100, about 95-105, about 100-110, about 105-115, about 110-120, about 115-125, about 120-130, about 125-135, about 130-140, or about 135-144 nucleotides. In some embodiments, the truncated AAV ITR has about 35, about 55, about 75, or about 95 nucleotides. In some embodiments, the truncated AAV ITR has about 75 nucleotides. In some embodiments, the truncated AAV ITR has about 35 nucleotides. In some embodiments, the truncated AAV ITR has about 55 nucleotides. In some embodiments, the truncated AAV ITR has about 95 nucleotides. In some embodiments, the truncated AAV ITR has 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, or 155 nucleotides.

In some embodiments, a truncated AAV ITR further comprises one or more nucleic acid mutations relative to a full-length AAV ITR of the same serotype. In some embodiments, the nucleic acid mutations are stabilizing mutations and/or result in more efficient ligation than a truncated AAV ITR which does not comprise said mutation(s). In some embodiments, the terminal nucleic acids (e.g., the 5′-most and 3′-most nucleic acids) of a truncated AAV ITR are mutated. In some embodiments, such mutation comprises the substitution of a terminal thymine (T) nucleic acid for a guanine (G) nucleic acid. In some embodiments, such mutation comprises the substitution of a terminal guanine (G) nucleic acid for a thymine (T) nucleic acid. In some embodiments, such mutation comprises the substitution of a terminal adenine (A) nucleic acid for a cytosine (C) nucleic acid. In some embodiments, such mutation comprises the substitution of a terminal cytosine (C) nucleic acid for an adenine (A) nucleic acid. In some embodiments, such mutation comprises the substitution of a terminal thymine (T) nucleic acid for a cytosine (C) nucleic acid. In some embodiments, such mutation comprises the substitution of a terminal cytosine (C) for a thymine (T) nucleic acid. In some embodiments, such mutation comprises the substitution of a terminal thymine (T) nucleic acid for an adenine (A) nucleic acid. In some embodiments, such mutation comprises the substitution of a terminal adenine (A) for a thymine (T) nucleic acid. In some embodiments, such mutation comprises the substitution of a terminal guanine (G) nucleic acid for a cytosine (C) nucleic acid. In some embodiments, such mutation comprises the substitution of a terminal cytosine (C) nucleic acid for a guanine (G) nucleic acid. In some embodiments, such mutation comprises the substitution of a terminal guanine (G) nucleic acid for an adenine (A) nucleic acid. In some embodiments, such mutation comprises the substitution of a terminal adenine (A) nucleic acid for a guanine (G) nucleic acid.

In some embodiments, the truncated AAV ITR is truncated relative to a full-length AAV ITR of the same serotype. Each AAV ITR in its full-length or wild-type form is about 145 nucleotides in length (e.g., about 140 nucleotides, about 145 nucleotides, about 150 nucleotides, about 155 nucleotides, about 160 nucleotides, or about 165 nucleotides). The AAV ITR sequence comprises a terminal sequence at the 5′ or 3′ end of the AAV genome which forms a palindromic double-stranded T-shaped hairpin structure, and an additional sequence which remains single-stranded (i.e., is not part of the T-shaped hairpin structure), termed the D-sequence.

In some embodiments, each recombinant AAV ITR can independently be an AAV ITR that is truncated relative to any AAV serotype (e.g., AAV1, AAV2, AAV3, AAV4, AAV5, AAV6, AAV7, AAV8, AAV9, AAV10, AAV11, AAV12, AAV13, AAVrh10, or AAVrh74). In other embodiments, both AAV ITRs may be of the same serotype. In some embodiments, the truncated AAV ITR is truncated relative to a full-length AAV ITR of AAV serotype 1, serotype 2, serotype 3, serotype 4, serotype 5, or serotype 6. In some embodiments, the truncated AAV ITR is truncated relative to a full-length AAV ITR of AAV serotype 1. In some embodiments, the truncated AAV ITR is truncated relative to SEQ ID NO: 1. In some embodiments, the truncated AAV ITR is truncated relative to a full-length AAV ITR of AAV serotype 2. In some embodiments, the truncated AAV ITR is truncated relative to SEQ ID NO: 2. In some embodiments, the truncated AAV ITR is truncated relative to a full-length AAV ITR of AAV serotype 3. In some embodiments, the truncated AAV ITR is truncated relative to SEQ ID NO: 3. In some embodiments, the truncated AAV ITR is truncated relative to a full-length AAV ITR of AAV serotype 4. In some embodiments, the truncated AAV ITR is truncated relative to SEQ ID NO: 4. In some embodiments, the truncated AAV ITR is truncated relative to a full-length AAV ITR of AAV serotype 5. In some embodiments, the truncated AAV ITR is truncated relative to SEQ ID NO: 5. In some embodiments, the truncated AAV ITR is truncated relative to a full-length AAV ITR of AAV serotype 6. In some embodiments, the truncated AAV ITR is truncated relative to SEQ ID NO: 6.

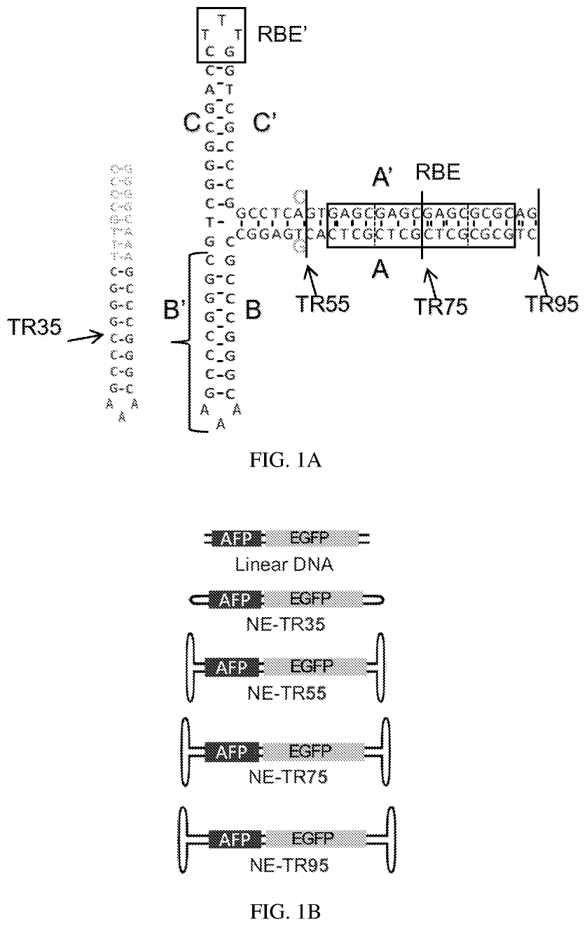

The present Examples demonstrate the truncation of an AAV2 ITR, relative to a full-length AAV ITR of AAV serotype 2 (see, e.g., FIG. 1A), which mediated significantly higher and more persistent transgene expression in no-end rAAV constructs than that which was observed in constructs not comprising the truncated AAV2 ITRs. However, the disclosure is not limited to those truncations nor those AAV serotypes exemplified by the present Examples, and truncated AAV ITRs of different lengths and/or of other AAV serotypes are specifically contemplated herein. One of skill in the art would understand, based on the present disclosure, how to construct truncated AAV ITRs of any AAV serotype, including but not limited to those AAV serotypes described herein, via, for example, the deletion of 5′ and/or 3′ AAV ITR nucleotides. Without wishing to being bound by theory, the truncated AAV ITRs of the present disclosure in some embodiments increase the expression level and/or increase the longevity of expression of a transgene, relative to a construct which does not comprise a truncated AAV ITR as described herein.

In some embodiments, the truncated AAV ITR is truncated by about 5 (e.g., about 5 nucleotides are deleted, relative to a full-length AAV ITR of the same serotype), about 10, about 20, about 30, about 35, about 40, about 45, about 50, about 55, about 60, about 65, about 70, about 75, about 80, about 85, about 90, about 95, about 100, about 105, about 110, or about 115 nucleotides, relative to a full-length AAV ITR of the same serotype (e.g., any one of SEQ ID NOs: 1-6). In some embodiments, the truncated AAV ITR is truncated by about 1-5, about 3-10, about 5-15, about 10-20, about 15-30, about 20-40, about 25-35, about 30-50, about 35-45, about 40-60, about 45-55, about 50-70, about 55-65, about 60-80, about 65-75, about 70-90, about 75-85, about 80-100, about 85-95, about 90-110, about 95-105, or about 100-120 nucleotides, relative to a full-length AAV ITR of the same serotype. In some embodiments, the truncated AAV ITR is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, or 115 nucleotides, relative to a full-length AAV ITR of the same serotype.

In some embodiments, truncating an AAV ITR comprises the deletion of nucleotides from the 5′ end of the AAV ITR sequence, and also deleting the corresponding complementary nucleotides from 3′ end of the AAV ITR sequence (e.g., as shown in FIG. 1A). In some embodiments, truncating an AAV ITR therefore comprises the deletion of a duplex structure within the AAV ITR sequence. In some embodiments, truncating an AAV ITR comprises any modification (e.g., a nucleotide deletion, substitution, mutation, or other disruption) which would de-stabilize the duplex structure of the AAV ITR (e.g., by preventing or disrupting Watson-Crick base pairing) while retaining the double-stranded nature of the no-end rAAV DNA. In some embodiments, truncating an AAV ITR comprises the deletion of nucleotides from the 5′ end of the AAV ITR sequence, and not deleting some or all of the corresponding complementary nucleotides from 3′ end of the AAV ITR sequence. In some embodiments, truncating an AAV ITR comprises the deletion of nucleotides from the 3′ end of the AAV ITR sequence, and not deleting some or all of the corresponding complementary nucleotides from 5′ end of the AAV ITR sequence.

TABLE 1
Full-length AAV ITRs of the disclosure.

| Description | Nucleotide Sequence | SEQ ID NO |
|---|---|---|
| Full-length AAV1 | TTGCCCACTCCCTCTCTGCGCGCTCGCTCGCTCGGTG | 1 |
| ITR (NCBI | GGGCCTGCGGACCAAAGGTCCGCAGACGGCAGAGCTC | |
| Reference Sequence: | TGCTCTGCCGGCCCCACCGAGCGAGCGAGCGCGCAGA | |
| NC_002077.1) | GAGGGAGTGGGCAACTCCATCACTAGGGGTAA | |
| Full-length AAV2 | TTGGCCACTCCCTCTCTGCGCGCTCGCTCGCTCACTG | 2 |
| ITR (e.g., ITR-145; | AGGCCGCCCGGGCAAAGCCCGGGCGTCGGGCGACCTT | |
| e.g., TR145) | TGGTCGCCCGGCCTCAGTGAGCGAGCGAGCGCGCAGA | |
| | GAGGGAGTGGCCAACTCCATCACTAGGGGTTCCT | |
| Full-length AAV3 | TTGGCCACTCCCTCTATGCGCACTCGCTCGCTCGGTG | 3 |
| ITR | GGGCCTGGCGACCAAAGGTCGCCAGACGGACGTGCTT | |
| | TGCACGTCCGGCCCCACCGAGCGAGCGAGTGCGCATA | |
| | GAGGGAGTGGCCAACTCCATCACTAGAGGTATGGC | |
| Full-length AAV4 | TTGGCCACTCCCTCTATGCGCGCTCGCTCACTCACTC | 4 |
| ITR (NCBI | GGCCCTGGAGACCAAAGGTCTCCAGACTGCCGGCCTC | |
| Reference Sequence: | TGGCCGGCAGGGCCGAGTGAGTGAGCGAGCGCGCATA | |
| NC_001829.1) | GAGGGAGTGGCCAACTCCATCATCTAGGTTTGCC | |
| Full-length AAV5 | CTCTCCCCCCTGTCGCGTTCGCTCGCTCGCTGGCTCG | 5 |
| ITR | TTTGGGGGGGTGGCAGCTCAAAGAGCTGCCAGACGAC | |
| | GGCCCTCTGGCCGTCGCCCCCCCAAACGAGCCAGCGA | |
| | GCGAGCGAACGCGACAGGGGGGAGAGTGCCACACTCT | |
| | CAAGCAAGGGGGTTTTGTA | |
| Full-length AAV6 | TTGCCCACTCCCTCTATGCGCGCTCGCTCGCTCGGTG | 6 |
| ITR | GGGCCTGCGGACCAAAGGTCCGCAGACGGCAGAGCTC | |
| | TGCTCTGCCGGCCCCACCGAGCGAGCGAGCGCGCATA | |
| | GAGGGAGTGGGCAACTCCATCACTAGGGGTA | |

TABLE 2
Truncated AAV2 ITRs of the disclosure.

| Description | Nucleotide Sequence (5′ to 3′) | SEQ ID NO |
|---|---|---|
| Truncated AAV2 ITR | GGGCAAAGGCCCGGGCAAAGCCCGGGCCTTTG | 7 |
| having 35 ITR nucleotides | CCC | |
| (e.g., ITR-35; e.g., TR-35) | GGGGCAAAGCCCGGGCAAAGCCCGGGCTTTGC | 11 |
| | CCC | |
| Truncated AAV2 ITR | GGAGGCCGCCCGGGCAAAGCCCGGGCGTCGGG | 8 |
| having 55 ITR nucleotides | CGACCTTTGGTCGCCCGGCCTCC | |
| (e.g., ITR-55; e.g., TR- | | |
| 55)* | | |
| Truncated AAV2 ITR | GCTCGCTCACTGAGGCCGCCCGGGCAAAGCCC | 9 |
| having 75 ITR nucleotides | GGGCGTCGGGCGACCTTTGGTCGCCCGGCCTC | |
| (e.g., ITR-75; e.g., TR-75) | AGTGAGCGAGC | |
| Truncated AAV2 ITR | CTGCGCGCTCGCTCGCTCACTGAGGCCGCCCG | 10 |
| having 95 ITR nucleotides | GGCAAAGCCCGGGCGTCGGGCGACCTTTGGTC | |
| (e.g., ITR-95; e.g., TR-95) | GCCCGGCCTCAGTGAGCGAGCGAGCGCGCAG | |

*In ITR-55, the terminal T and A nucleotides were changed to G and C (bold, underlined), respectively, to make the ends more stable and for efficient ligation.

In some embodiments, the recombinant AAV ITR (e.g., truncated AAV ITR) comprises a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleotide sequence of any one of SEQ ID NOs: 7-11. In some embodiments, the recombinant AAV ITR (e.g., truncated AAV ITR) comprises the nucleotide sequence of any one of SEQ ID NOs: 7-11.

In some embodiments, a recombinant AAV ITR (e.g., truncated AAV ITR) comprises a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleotide sequence of SEQ ID NO: 9. In some embodiments, the recombinant AAV ITR (e.g., truncated AAV ITR) comprises the nucleotide sequence of SEQ ID NO: 9.

In some embodiments, a recombinant AAV ITR (e.g., truncated AAV ITR) comprises a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleotide sequence of SEQ ID NO: 7. In some embodiments, the recombinant AAV ITR (e.g., truncated AAV ITR) comprises the nucleotide sequence of SEQ ID NO: 7.

In some embodiments, a recombinant AAV ITR (e.g., truncated AAV ITR) comprises a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleotide sequence of SEQ ID NO: 8. In some embodiments, the recombinant AAV ITR (e.g., truncated AAV ITR) comprises the nucleotide sequence of SEQ ID NO: 8.

In some embodiments, a recombinant AAV ITR (e.g., truncated AAV ITR) comprises a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleotide sequence of SEQ ID NO: 10. In some embodiments, the recombinant AAV ITR (e.g., truncated AAV ITR) comprises the nucleotide sequence of SEQ ID NO: 10.

In some embodiments, a recombinant AAV ITR (e.g., truncated AAV ITR) comprises a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleotide sequence of SEQ ID NO: 11. In some embodiments, the recombinant AAV ITR (e.g., truncated AAV ITR) comprises the nucleotide sequence of SEQ ID NO: 11.

### Compositions

Aspects of the invention include a composition comprising a no-end rAAV genome as described herein. Conventional viral and non-viral based gene transfer methods can be used to introduce nucleic acids into cells (e.g., mammalian cells) and target tissues. Viral vector delivery systems include DNA and RNA viruses, which have either episomal or integrated genomes after delivery to the cell. Non-viral vector delivery systems include DNA plasmids, naked nucleic acids, and nucleic acids complexed with a delivery vehicle such as a liposome or poloxamer. Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. In some embodiments, the composition comprises an AAV particle, a liposome, a nanoparticle, or an exosome. An exemplary liposome for the delivery of NE-rAAV is shown in FIG. 5A.

In some embodiments, the composition comprises an AAV particle. In some embodiments, an AAV particle comprises an empty capsid (e.g., a capsid without a cargo). In some embodiments, an AAV particle comprises a capsid encapsidating a nucleic acid (e.g., a no-end rAAV that comprises a gene of interest). In some embodiments, a nucleic acid encapsidated within an AAV capsid to generate an AAV particle comprises a no-end rAAV genome as disclosed herein. In some embodiments, an AAV particle disclosed herein comprises a capsid protein comprising one or more mutations, e.g., one or more amino acid substitutions.

In some embodiments, an AAV particle disclosed herein is replicative. A replicative AAV particle is capable of replicating within a host cell (e.g., a host cell within a subject or a host cell in culture). In some embodiments, an AAV particle disclosed herein is non-replicating. A non-replicating AAV particle is not capable of replicating within a host cell (e.g., a host cell within a subject or a host cell in culture), but can infect the host and incorporate genetic components into the host's genome for expression. In some embodiments, an AAV particle disclosed herein is capable of infecting a host cell. In some embodiments, an AAV particle disclosed herein is capable of facilitating stable integration of genetic components into the genome of a host cell. In some embodiments, an AAV particle disclosed herein is not capable of facilitating integration of genetic components into the genome of a host cell.

In some embodiments, an AAV particle disclosed herein comprises a no-end rAAV genome of the disclosure. In some embodiments, as described elsewhere herein, a no-end rAAV genome comprises two AAV ITRs flanking the ends of a sequence encoding a heterologous gene of interest. In some embodiments, the no-end rAAV genome is comprised within the AAV particle's single-stranded (ss) DNA genome. In some embodiments, an AAV particle disclosed herein comprises one single-stranded DNA. In some embodiments, an AAV particle disclosed herein comprises two complementary DNA strands, forming a self-complementary AAV (scAAV).

An AAV particle disclosed herein may be of any AAV serotype (e.g., AAV serotype 1, 2, 3, 4, 5, 6, 7, 8, 9, 10, 11, 12, or 13), including any derivative (including non-naturally occurring variants of a serotype) or pseudotype. Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV2.5T, AAV-HAE1/2, AAV clone 32/83, AAVShH10, AAV2.15, AAV2.4, AAVM41, and AAVr3.45. Such AAV serotypes and derivatives/pseudotypes, and methods of producing such derivatives/pseudotypes are known in the art (scc, e.g., Asokan, et al. Mol. Ther. 2012 April; 20(4):699-708). In some embodiments, the AAV particle is a pseudotyped AAV particle, which comprises a nucleic acid vector comprising AAV ITRs from one serotype (e.g., AAV2 or AAV3) and a capsid comprised of capsid proteins derived from another serotype (i.e., a serotype other than AAV2 or AAV3, respectively). Methods for producing and using pseudotyped rAAV vectors are known in the art (sec, e.g., Duan et al., J. Virol., 75:7662-7671 (2001); Halbert et al., J. Virol., 74:1524-1532 (2000); Zolotukhin et al., Methods, 28:158-167 (2002); and Auricchio et al., Hum. Molec. Genet., 10:3075-3081 (2001)). In some embodiments, the AAV particle is an AAV serotype 1, 2, 3, 4, 5, 6, 7, 8, 9, 10, 11, 12, or 13 particle.

In some embodiments, an AAV particle disclosed herein is a recombinant AAV (rAAV) particle, e.g., comprising a recombinant nucleic acid or transgene.

In some embodiments, a no-end rAAV genome is formulated in a composition comprising a liposome. A liposome is a spherical-shaped vesicle that is composed of one or more phospholipid bilayers, which closely resembles the structure of cell membranes. The ability of liposomes to encapsulate hydrophilic or lipophilic drugs have allowed these vesicles to become useful drug delivery systems. Recently, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a product of interest (see Feigner, et. al., Proc. Natl. Acad. Sci. U.S.A. 84:7413-7417 (1987); Mackey, et al., Proc. Natl Acad. Sci. U.S.A. 85:8027-8031 (1988); Ulmer et al., Science 259:1745-1748 (1993)). The use of cationic lipids may promote encapsulation of negatively charged nucleic acids, and may also promote fusion with negatively charged cell membranes (see Feigner and Ringold, Science 337:387-388 (1989)). Particularly useful lipid compounds and compositions for transfer of nucleic acids are described in International Patent Publications WO95/18863 and WO96/17823, and in U.S. Pat. No. 5,459,127. Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931). In some embodiments, the liposome comprises a galactosylated lipid.

In some embodiments, a no-end rAAV genome is formulated in a composition comprising a nanoparticle. In some embodiments, a no-end rAAV genome is formulated in a composition comprising a lipid nanoparticle. In some embodiments, a no-end rAAV genome is formulated in a composition comprising a lipid-polycation complex, referred to as a cationic lipid nanoparticle. As a non-limiting example, the polycation may include a cationic peptide or a polypeptide such as, but not limited to, polylysine, polyomithine, and/or polyarginine. In some embodiments a no-end rAAV genome is formulated in a composition comprising a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components, and biophysical parameters such as size. In one example by Semple, et al. (Nature Biotech. 2010, 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (scc, e.g., Basha, et al., Mol Ther. 2011, 19:2186-2200).

In some embodiments, lipid nanoparticle formulations may comprise 35 to 45% cationic lipid, 40% to 50% cationic lipid, 50% to 60% cationic lipid, and/or 55% to 65% cationic lipid.

In some embodiments, a no-end rAAV genome is formulated in a composition comprising a nanoparticle that comprises at least one lipid. The lipid may be selected from, but is not limited to, DLin-DMA, DLin-K-DMA, 98N12-5, C 12-200, DLin-MC3-DMA, DLin-KC2-DMA, DODMA, PLGA, PEG, PEG-DMG, PEGylated lipids, and amino alcohol lipids. In some embodiments, the lipid may be a cationic lipid such as, but not limited to, DLin-DMA, DLin-D-DMA, DLin-MC3-DMA, DLin-KC2-DMA, DODMA, and amino alcohol lipids.

The amino alcohol cationic lipid may be the lipids described in and/or made by the methods described in U.S. Patent Publication No. US20130150625, for example. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1.3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol, and a molecule capable of reducing particle aggregation, for example a PEG or PEG-modified lipid.

In some embodiments, a no-end rAAV genome is formulated in a composition comprising an exosome. In some embodiments, the exosome comprises an exosome-associated rAAV. Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome and exhibit membrane proteins from the parental cell on the surface. An exemplary exosome system for delivering NE-rAAV is shown in FIG. 5B.

Exosomes exhibit different compositions and functions depending on the cell type from which they are derived. There are no “exosome-specific” proteins; however, several proteins identified in these vesicles are associated with endosomes and lysosomes reflecting their origin. Most exosomes are enriched in MHC I and II (major histocompatibility complex I and II; important for antigen presentation to immunocompetent cells such as T-lymphocytes), tetraspanins, several heat shock proteins, cytoskeletal components such as actins and tubulins, proteins involved in intracellular membrane fusion, signal transduction proteins, and cytosolic enzymes.

Exosomes are produced by many cells including epithelial cells, B and T lymphocytes, mast cells (MC), and dendritic cells (DC). In humans, exosomes have been found in blood, plasma, urine, broncho alveolar lavage fluid, intestinal epithelial cells, and tumor tissues.

Aspects of the disclosure include the use of exosomes to identify certain peptide antigens and/or immunogens that are specific to the subject being administered the no-end rAAV genome or composition comprising a no-end rAAV genome of the present disclosure. In some embodiments, subject-specific peptide antigens and/or immunogens of a no-end rAAV genome are identified in an exosome of the subject. When peptide antigens are identified in an exosome of the subject, such antigens are said to be representative of exosome antigens of the subject.

A number of methods of isolating exosomes from a biological sample have been described in the art. For example, the following methods can be used: differential centrifugation, low speed centrifugation, anion exchange and/or gel permeation chromatography, sucrose density gradients or organelle electrophoresis, magnetic activated cell sorting (MACS), nanomembrane ultrafiltration concentration, Percoll gradient isolation, and/or using microfluidic devices. Exemplary methods are described in U.S. Patent Publication No. 2014/0212871, for example.

Methods of producing exosome-associated rAAV, which can be more resistant to neutralizing anti-AAV antibodies, are also known (Hudry, et al., Gene Ther. 2016, 23(4):380-92; Macguire, et al., Mol Ther. 2012, 20(5):960-71).

### Methods

Aspects of the invention include a method of treating a disease or disorder, the method comprising administering a no-end rAAV genome as described herein or a composition comprising a no-end rAAV genome as described herein to a subject in need thereof. In some embodiments, the subject is a mammal. In some embodiments, the subject is a human, non-human primate, dog, cat, pig, mouse, horse, sheep, goat, rat, guinea pig, hamster, or cow. In some embodiments, the subject is a human. In some embodiments, the subject is a human child (e.g., a human being less than 18 years of age).

In some embodiments, “administering” or “administration” means providing a material to a subject in a manner that is pharmacologically useful. In some embodiments, a no-end rAAV genome as described herein or a composition comprising a no-end rAAV genome as described herein is administered to a subject enterally. In some embodiments, an enteral administration of the no-end rAAV genome as described herein or the composition comprising a no-end rAAV genome as described herein is oral. In some embodiments, a no-end rAAV genome as described herein or a composition comprising a no-end rAAV genome as described herein is administered to the subject parenterally. In some embodiments, a no-end rAAV genome as described herein or a composition comprising a no-end rAAV genome as described herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a no-end rAAV genome as described herein or a composition comprising a no-end rAAV genome as described herein is administered to the subject by injection into the hepatic artery or portal vein. In some embodiments, the no-end rAAV genome or the composition is administered intramuscularly, intravenously, subcutaneously, intrathecally, intraperitoneally, or by direct injection into an organ or a tissue of the subject.

Any one of the AAV particles, capsid proteins, or nucleic acids disclosed herein may be comprised within a pharmaceutical composition comprising a pharmaceutically-acceptable carrier or may be comprised within a pharmaceutically-acceptable carrier. The term “carrier” refers to a diluent, adjuvant, excipient, or vehicle with which the AAV particle, capsid protein, or nucleic acid is comprised or administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases), and solutions or compositions thereof. Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urca, alcohols, ascorbic acid, phospholipids, proteins (for example, serum albumin), EDTA, sodium chloride, liposomes, mannitol, sorbitol, and glycerol. USP grade carriers and excipients are particularly useful for delivery of AAV particles to human subjects.

Typically, such compositions may contain at least about 0.1% of the therapeutic agent (e.g., AAV particle) or more, although the percentage of the active ingredient(s) may, of course, be varied and may be between about 1 or 2% and about 70% or 80% or more of the weight or volume of the total formulation. Naturally, the amount of therapeutic agent(s) (e.g., AAV particle) in each therapeutically-useful composition may be prepared in such a way that a suitable dosage will be obtained in any given unit dose of the compound. Factors such as solubility, bioavailability, biological half-life, route of administration, and product shelf life, as well as other pharmacological considerations, will be contemplated by one skilled in the art of preparing such pharmaceutical formulations, and as such, a variety of dosages and treatment regimens may be designed.

In some embodiments, a no-end rAAV genome as described herein or a composition comprising a no-end rAAV genome as described herein is administered to a subject to treat a disease or condition. To “treat” a disease, as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of no-end rAAV particles may be an amount of the particles that are capable of transferring an expression construct to a host organ, tissue, or cell. A therapeutically acceptable amount may be an amount that is capable of treating a disease, e.g., a hemophilia. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In embodiments wherein a no-end rAAV genome of the present disclosure is comprised in a composition comprising an AAV particle, the concentration of AAV particles administered to a subject may be on the order ranging from 10⁶ to 10¹⁴ particles/ml or 10³ to 10¹⁵ particles/ml, or any values therebetween for either range, such as for example, about 10³, 10⁴, 10⁵, 10⁶, 10⁷, 10⁸, 10⁹, 10¹⁰, 10¹¹, 10¹², 10¹³, 10¹⁴, or 10¹⁵ particles/ml. In some embodiments, AAV particles of a higher concentration than 10¹³ particles/ml are administered. In some embodiments, the concentration of AAV particles administered to a subject may be on the order ranging from 10⁶ to 10¹⁴ vector genomes (vgs)/ml or 10³ to 10¹⁵ vgs/ml, or any values therebetween for either range (e.g., 10³, 10⁴, 10⁵, 10⁶, 10⁷, 10⁸, 10⁹, 10¹⁰, 10¹¹, 10¹², 10¹³, 10¹⁴, or 10¹⁵ vgs/ml). In some embodiments, AAV particles of higher concentration than 10¹³ vgs/ml are administered. The AAV particles can be administered as a single dose, or divided into two or more administrations as may be required to achieve therapy of the particular disease or disorder being treated. In some embodiments, 0.0001 ml to 10 ml are delivered to a subject. In some embodiments, the number of AAV particles administered to a subject may be on the order ranging from 10⁶-10¹⁴ vgs/kg body mass of the subject, or any values therebetween (e.g., 10⁶, 10⁷, 10⁸, 10⁹, 10¹⁰, 10¹¹, 10¹², 10¹³, or 10¹⁴ vgs/kg). In some embodiments, the dose of AAV particles administered to a subject may be on the order ranging from 10¹²-10¹⁴ vgs/kg. In some embodiments, the volume of a composition comprising an AAV particle delivered to a subject (e.g., via one or more routes of administration as described herein) is 0.0001 ml to 10 ml.

In some embodiments, a composition disclosed herein (e.g., comprising an AAV particle, liposome, nanoparticle, or exosome) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject.

In some embodiments, the subject has or is suspected of having a disease or disorder that may be treated with gene therapy. In some embodiments, the subject has or is suspected of having a disease or disorder associated with the liver, or cells comprised therein. In some embodiments, a nucleic acid isolated or derived from the subject (e.g., genomic DNA, mRNA, or cDNA from the subject) is identified via sequencing (e.g., Sanger or next-generation sequencing) to comprise a mutation (e.g., in a gene associated with muscle development, health, maintenance, or function). In some embodiments, the disease or disorder is selected from the group consisting of: Alpha 1-Antitrypsin Deficiency, Phenylketonuria, Wilson Disease, Acute Intermittent Porphyria, Familial Hypercholesterolemia, Crigler-Najjar Syndrome, ATTR Amyloidosis, Methylmalonic Acidemia, a Mucopolysaccharidosis, Glycogen Storage Disease Type Ia. Ornithine Transcarbamylase Deficiency, liver fibrosis, Hemophilia A, Hemophilia B, and Hemophilia C. In some embodiments, the disease or disorder is selected from the group consisting of: liver fibrosis, Hemophilia A, Hemophilia B, and Hemophilia C.

In some embodiments, a no-end rAAV genome as described herein or a composition comprising a no-end rAAV genome as described herein is administered to a subject in need thereof, and, following administration, AAV Rep proteins mediate site-specific integration of therapeutic genes into human chromosome 19 [19q13.3]. In some embodiments, such site-specific integration into 19q13.3 is useful for the treatment of a liver-associated disease, as described herein.

In some embodiments, the method comprises contacting a cell with a no-end rAAV genome as described herein or a composition comprising a no-end rAAV genome as described herein. In some embodiments, a cell disclosed herein is a cell isolated or derived from a subject. In some embodiments, a cell is a mammalian cell (e.g., a cell isolated or derived from a mammal). In some embodiments, the cell is a human cell, non-human primate cell, rat cell, or mouse cell. In some embodiments, a cell is isolated or derived from a particular tissue of a subject, such as liver tissue. In some embodiments, the cell is a liver, brain, heart or retina cell. In some embodiments, a cell is a liver cell. In some embodiments, a cell is in vitro. In some embodiments, a cell is ex vivo. In some embodiments, a cell in in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

Methods of contacting a cell may comprise, for example, contacting a cell in a culture with a no-end rAAV genome as described herein or a composition comprising a no-end rAAV genome as described herein. In some embodiments, contacting a cell comprises adding a no-end rAAV genome as described herein or a composition comprising a no-end rAAV genome as described herein to the supernatant of a cell culture (e.g., a cell culture on a tissue culture plate or dish) or mixing a no-end rAAV genome as described herein or a composition comprising a no-end rAAV genome as described herein with a cell culture (e.g., a suspension cell culture). In some embodiments, contacting a cell comprises mixing a no-end rAAV genome as described herein or a composition comprising a no-end rAAV genome as described herein with another solution, such as a cell culture media, and incubating a cell with the mixture.

In some embodiments, contacting a cell with a no-end rAAV genome as described herein or a composition comprising a no-end rAAV genome as described herein comprises administering a no-end rAAV genome as described herein or a composition comprising a no-end rAAV genome as described herein to a subject or device in which the cell is located. In some embodiments, contacting a cell comprises injecting a no-end rAAV genome as described herein or a composition comprising a no-end rAAV genome as described herein into a subject in which the cell is located. In some embodiments, contacting a cell comprises administering a no-end rAAV genome as described herein or a composition comprising a no-end rAAV genome as described herein directly to a cell, or into or substantially adjacent to a tissue of a subject in which the cell is present.

### ENUMERATED EMBODIMENTS

Certain embodiments are set forth in the enumerated clauses below.

Clause 1. A no-end (NE) recombinant adeno-associated virus (rAAV) genome comprising a heterologous gene flanked by recombinant inverted terminal repeats (ITRs), wherein at least one of the recombinant ITRs is a truncated ITR having between about 35 and 95 ITR nucleotides, wherein the truncated ITR is truncated relative to a full-length AAV ITR of the same serotype.

Clause 2. The no-end rAAV genome of clause 1, wherein the truncated ITR has about 55-95 nucleotides.

Clause 3. The no-end rAAV genome of clause 1, wherein the truncated ITR has about 35, about 55, about 75, or about 95 nucleotides.

Clause 4. The no-end rAAV genome of any one of clauses 1-3, wherein the truncated ITR has about 75 nucleotides.

Clause 5. The no-end rAAV genome of clause 1 or clause 3, wherein the truncated ITR has about 35 nucleotides.

Clause 6. The no-end rAAV genome of any one of clauses 1-3, wherein the truncated ITR has about 55 nucleotides.

Clause 7. The no-end rAAV genome of any one of clauses 1-3, wherein the truncated ITR has about 95 nucleotides.

Clause 8. The no-end rAAV genome of any one of clauses 1-7, wherein the no-end rAAV genome does not comprise a nucleotide sequence capable of encoding AAV capsid protein(s).

Clause 9. The no-end rAAV genome of any one of clauses 1-8, wherein the truncated ITR further comprises one or more nucleic acid mutations relative to a full-length AAV ITR of the same serotype.

Clause 10. The no-end rAAV genome of clause 9, wherein the nucleic acid mutations are stabilizing mutations and/or result in more efficient ligation than a truncated ITR which does not comprise said mutation(s).

Clause 11. The no-end rAAV genome of any one of clauses 1-10, wherein the truncated ITR is truncated relative to a full-length ITR of AAV serotype 1, 2, 3, 4, 5, or 6.

Clause 12. The no-end rAAV genome of any one of clauses 1-11, wherein the truncated ITR is truncated relative to a full-length ITR of AAV serotype 2.

Clause 13. The no-end rAAV genome of any one of clauses 1-12, wherein the truncated ITR is truncated relative to SEQ ID NO: 2.

Clause 14. The no-end rAAV genome of any one of clauses 1-13, wherein the truncated ITR comprises a nucleotide sequence having at least 90%, at least 95%, at least 98%, or at least 99% identity to the nucleotide sequence of any one of SEQ ID NOs: 7-11.

Clause 15. The no-end rAAV genome of any one of clauses 1-14, wherein the truncated ITR comprises the nucleotide sequence of any one of SEQ ID NOs: 7-11.

Clause 16. The no-end rAAV genome of any one of clauses 1-15, wherein the truncated ITR comprises the nucleotide sequence of SEQ ID NO: 9.

Clause 17. The no-end rAAV genome of any one of clauses 1-15, wherein the truncated ITR comprises the nucleotide sequence of SEQ ID NO: 7 or SEQ ID NO: 11.

Clause 18. The no-end rAAV genome of any one of clauses 1-15, wherein the truncated ITR comprises the nucleotide sequence of SEQ ID NO: 8.

Clause 19. The no-end rAAV genome of any one of clauses 1-15, wherein the truncated ITR comprises the nucleotide sequence of SEQ ID NO: 10.

Clause 20. The no-end rAAV genome of any one of clauses 1-19, wherein the heterologous gene is a gene associated with one or more of the following diseases or disorders: Alpha 1-Antitrypsin Deficiency, Phenylketonuria, Wilson Disease, Acute Intermittent Porphyria, Familial Hypercholesterolemia, Crigler-Najjar Syndrome, ATTR Amyloidosis, Methylmalonic Acidemia, a Mucopolysaccharidosis, Glycogen Storage Disease Type Ia, Ornithine Transcarbamylase Deficiency, liver fibrosis, Hemophilia A, Hemophilia B, and Hemophilia C.

Clause 21. The no-end rAAV genome of any one of clauses 1-20, wherein the heterologous gene is a gene associated with one or more of the following diseases or disorders: liver fibrosis, Hemophilia A, Hemophilia B, and Hemophilia C.

Clause 22. The no-end rAAV genome of any one of clauses 1-21, wherein the heterologous gene encodes albumin, Factor VIII (F.VIII), or Factor IX (F.IX).

Clause 23. The no-end rAAV genome of any one of clauses 1-22, further comprising a promoter.

Clause 24. The no-end rAAV genome of clause 23, wherein the promoter is a liver-specific promoter and/or a liver cancer cell-specific promoter.

Clause 25. The no-end rAAV genome of clause 23 or clause 24, wherein the promoter comprises any one of: human liver cell-specific trans-thyretin (TTR) promoter, human α-fetoprotein promoter (AFP), human al-antitrypsin (hAAT) promoter, human F.IX promoter, human F.VIII promoter, and liver promoter 1 (LP1).

Clause 26. The no-end rAAV genome of any one of clauses 1-25, wherein both recombinant ITRs are truncated relative to a full-length AAV ITR of the same serotype.

Clause 27. A composition comprising the no-end rAAV genome of any one of clauses 1-26.

Clause 28. The composition of clause 27, wherein the composition comprises an AAV particle, a liposome, a nanoparticle, or an exosome.

Clause 29. The composition of clause 28, wherein the AAV particle is an AAV serotype 1, 2, 3, 4, 5, 6, 7, 8, 9, 10, 11, 12, or 13 particle.

Clause 30. The composition of clause 28, wherein the nanoparticle comprises a lipid nanoparticle.

Clause 31. The composition of clause 28, wherein the exosome comprises an exosome-associated rAAV.

Clause 32. A method of treating a disease or disorder, the method comprising administering the no-end rAAV genome of any one of clauses 1-26 or the composition of any one of clauses 27-31 to a subject in need thereof.

Clause 33. The method of clause 32, wherein the subject is a human.

Clause 34. The method of clause 32 or clause 33, wherein the subject is a human child.

Clause 35. The method of any one of clauses 32-34, wherein the no-end rAAV genome or the composition is administered intramuscularly, intravenously, subcutaneously, intrathecally, intraperitoneally, or by direct injection into an organ or a tissue of the subject.

Clause 36. The method of any one of clauses 32-35, wherein the disease or disorder is selected from the group consisting of: Alpha 1-Antitrypsin Deficiency, Phenylketonuria, Wilson Disease, Acute Intermittent Porphyria, Familial Hypercholesterolemia, Crigler-Najjar Syndrome, ATTR Amyloidosis, Methylmalonic Acidemia, a Mucopolysaccharidosis, Glycogen Storage Disease Type Ia, Ornithine Transcarbamylase Deficiency, liver fibrosis, Hemophilia A, Hemophilia B, and Hemophilia C.

Clause 37. The method of any one of clauses 32-36, wherein the disease or disorder is selected from the group consisting of: liver fibrosis, Hemophilia A, Hemophilia B, and Hemophilia C.

Clause 38. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 9.

Clause 39. The recombinant AAV ITR of clause 38, wherein the recombinant AAV ITR comprises the nucleotide sequence of SEQ ID NO: 9.

Clause 40. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 7.

Clause 41. The recombinant AAV ITR of clause 40, wherein the recombinant AAV ITR comprises the nucleotide sequence of SEQ ID NO: 7.

Clause 42. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 8.

Clause 43. The recombinant AAV ITR of clause 42, wherein the recombinant AAV ITR comprises the nucleotide sequence of SEQ ID NO: 8.

Clause 44. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 10.

Clause 45. The recombinant AAV ITR of clause 44, wherein the recombinant AAV ITR comprises the nucleotide sequence of SEQ ID NO: 10.

Clause 46. The no-end rAAV genome of any one of clauses 1-26 or the composition of any one of clauses 27-31, further comprising a nucleotide sequence encoding an AAV Rep protein.

Clause 47. The no-end rAAV genome or composition of clause 46, wherein the heterologous gene is integrated into human chromosome 19 (19q13.3).

Clause 48. The no-end rAAV genome of any one of clauses 1-26 or 46-47, wherein the heterologous gene comprises a: Serpin Family A Member 1 (SERPINA1) gene, gene encoding Phenylalanine Hydroxylase (PAH), ATPase Copper Transporting Beta (ATPB7) gene, Hydroxymethylbilane Synthase (HMBS) gene, low density lipoprotein receptor (LDLR) gene, UDP Glucuronosyltransferase Family 1 Member A Complex Locus (UGTIA) gene, transthyretin (TTR) gene, methylmalonyl Coenzyme A mutase (MMUT) gene, Mucopolysaccharidosis Type I (MPS I) gene, Mucopolysaccharidosis Type II (MPS II) gene, Mucopolysaccharidosis Type IIIA (MPS IIIA) gene, Mucopolysaccharidosis Type IIIB (MPS IIIB) gene, Mucopolysaccharidosis Type IIIC (MPS IIIC) gene, Mucopolysaccharidosis Type IIID (MPS IIID) gene, Mucopolysaccharidosis Type IVA (MPS IVA) gene, Mucopolysaccharidosis Type IVB (MPS IVB) gene, Mucopolysaccharidosis Type V (MPS V) gene, Mucopolysaccharidosis Type VI (MPS VI) gene, Mucopolysaccharidosis Type VII (MPS VII) gene, Mucopolysaccharidosis Type IX (MPS IX) gene, glucose-6-phosphatase catalytic-subunit (G6PC) gene, Ornithine Transcarbamylase (OTC) gene, gene encoding human serum albumin, gene encoding clotting Factor VIII (F.VIII), or gene encoding clotting Factor IX (F.IX).

Clause 49. The no-end rAAV genome of any one of clauses 1-26 or 46-48, wherein the heterologous gene encodes: α-1 anti-trypsin protein, phenylalanine hydroxylase, ATPase Copper Transporting Beta, porphobilinogen deaminase, Hydroxymethylbilane Synthase, low-density lipoprotein receptor, uridine diphosphoglucuronate glucuronosyltransferase, transthyretin protein, methylmalonyl Coenzyme A mutase, α-L-iduronidase, iduronate sulfatase, heparan N-sulfatase, α-N-acetylglucosaminidase, acetyl-CoA α-glucosaminide acetyltransferase, N-acetylglucosamine 6-sulfatase, N-acetylgalactosamine-6-sulfatase, β-galactosidase, α-L-iduronidase, N-acetylgalactosamine 4-sulfatase, β-glucuronidase, hyaluronidase, glucose-6-phosphatase, ornithine Transcarbamylase, human serum albumin, clotting Factor VIII (F.VIII), or clotting Factor IX (F.IX).

### EXAMPLES

Recombinant AAV serotype vectors and their variants have been, or are currently being, used for gene therapy for hemophilia in several Phases I/II/III clinical trials in adult patients. However, none of these trials have included children with hemophilia, since the traditional liver-directed AAV gene therapy will not work in these patients. Such traditional therapy will not work in children because: (i) Up until age 10-12, the liver is still growing and dividing, and with every cell division the AAV vector genomes will be diluted out due to their episomal nature; and (ii) Repeated gene delivery will be needed, but repeat dosing, even with an ideal AAV vector, is not an option because of the development of pre-existing antibodies to AAV vectors following the first administration.

The use and development of a synthetic AAV vector, devoid of AAV capsid proteins and capable of repeat dosing, is disclosed herein. A novel No-End (NE) AAV DNA was developed based upon a previously-described hybrid genome that consists of a gene cassette covalently flanked by AAV inverted terminal repeats (ITRs) (see, e.g., FIG. 1A) with no open ends. Although this linear, NE-DNA is exonuclease-resistant, and can be used to express a transgene following delivery into mammalian cells, the stability of NE-DNA and kinetics of transgene expression have not been studied in detail.

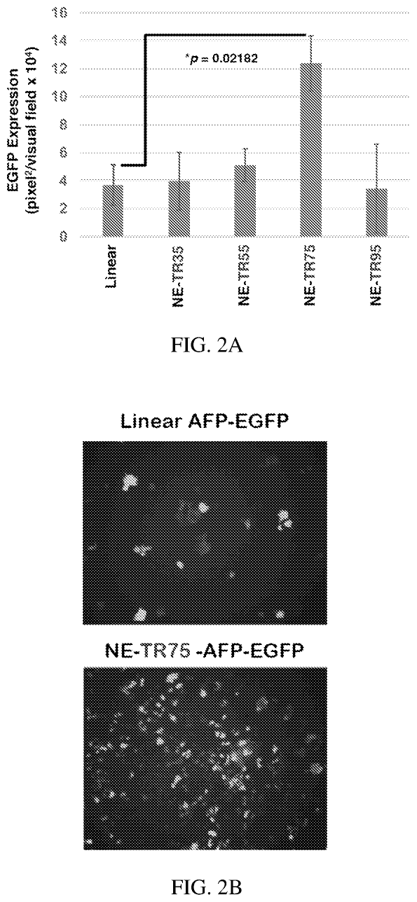

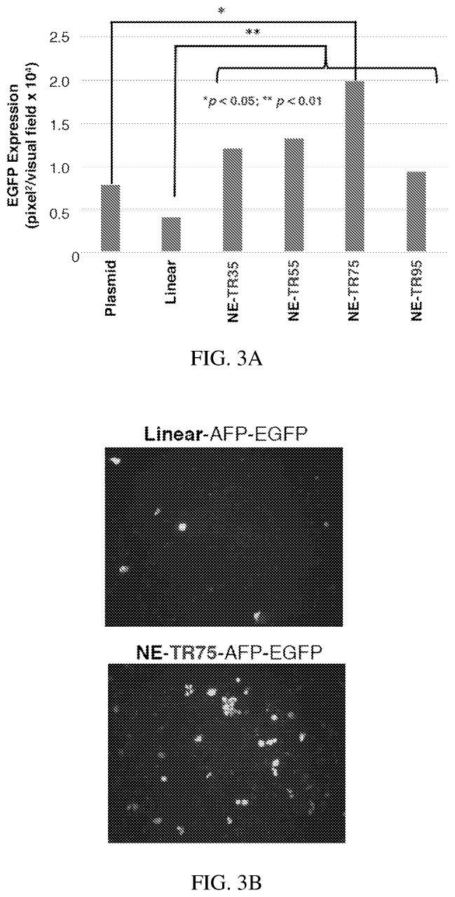

In addition to the full-length, 145-bp AAV ITRs, several NE-DNAs with truncated AAV ITRs (ranging from 35-95 bps) were constructed and assessed for transgene expression and longevity from an enhanced green fluorescent protein (EGFP) expression cassette under the control of the human α-fetoprotein (AFP) promoter (FIG. 1B). It was observed that transgene expression mediated by each of the NE-DNAs is significantly higher than that from either the plasmid or the linear DNAs up to 28 days post-transfection. An optimal NE-DNA construct (NE-TR75) was found to mediate transgene expression in human hepatocellular carcinoma cell line, Huh7, for up to 4-weeks post-transfection (FIGS. 3A and 3B). An effect was also observed as early as 3 days post-transfection (FIGS. 2A and 2B).

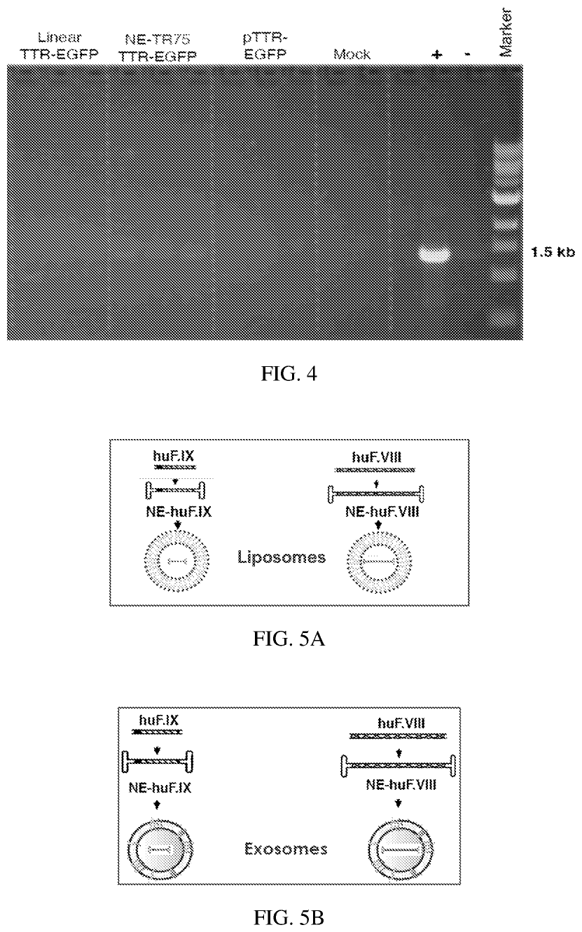

At 4-weeks post-transfection, the transgene expression half-life of NE-TR75 DNA was observed to be ˜1.6-fold longer than linear DNA (˜9.8 days for NE-TR75 DNA compared to ˜6.1 days for linear DNA) and ˜1.8-fold longer than circular plasmid DNA (˜9.8 days for NE-TR75 DNA compared to ˜5.3 days for circular plasmid DNA) (FIGS. 3A and 3B). The longevity of transgene expression also correlated well with the stability of the NE-TR75 DNA construct containing the EGFP expression cassette under the control of the human liver cell-specific trans-thyretin (TTR) promoter up to 35 days post-transfection, as determined by PCR analysis of extrachromosomal DNA (FIG. 4).

However, it is unlikely that naked DNA, including NE-DNA, will be able to escape degradation following systemic delivery. To address this, other NE-DNAs containing human clotting factors IX (hF.IX) and VIII (hF.VIII) were also developed, such as NE-TR75-human clotting factors IX (hF.IX) and VIII (hF.VIII) DNAs under the control of the TTR promoter (FIGS. 5A and 5B).

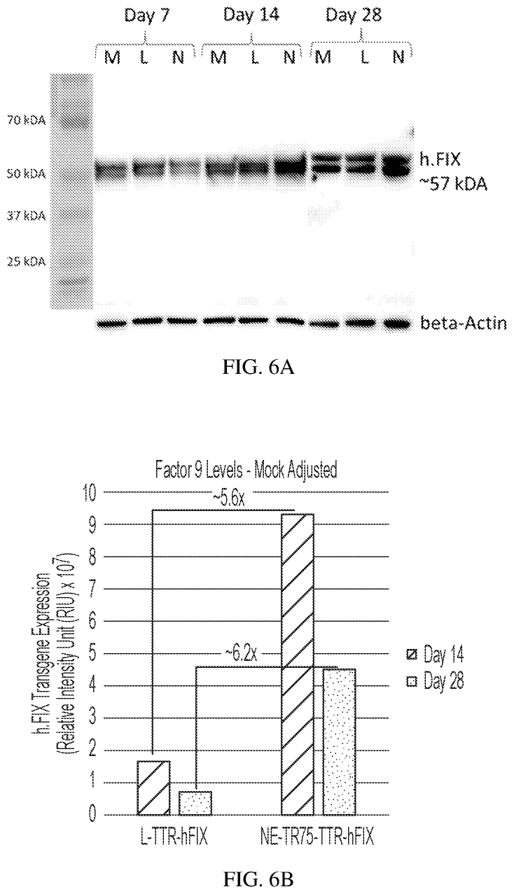

Studies were performed to evaluate the efficacy and longevity of hF.IX and hF. VIII expression in human hepatic cells. FIGS. 6A and 6B show the increased expression of human Factor IX (hF.IX) from NE-TR75 TTRp-hF.IX DNA in a human liver cell line (HepG2 cells) in vitro. FIG. 6A shows western blot analysis of hF.IX expression in human HepG2 cells 7 days, 14 days, and 28 days following mock-transfection (M), transfection with linear-TTRp-hF.IX (L), or transfection with NE-TR75-TTRp-hF.IX (N) DNA using monoclonal anti-hF.IX antibody. FIG. 6B shows quantitation of the data from FIG. 6A for 14 and 28 days post-transfection. At 14 days post-transfection, a ˜5.6× increase in h.FIX transgene expression was observed when cells were transfected with NE-TR75-TTR-hF.IX, rather than with L-TTR-hF.IX. At 28 days post-transfection, a ˜6.2× increase in h.FIX transgene expression was observed when cells were transfected with NE-TR75-TTR-hF.IX, rather than with L-TTR-hF.IX.

The NE-DNA constructs disclosed herein are encapsulated in liposomes. Such encapsidation will result in liver-specific delivery and high-efficiency, long-term transgene expression. In some embodiments, the AAV Rep gene is included in these NE-DNAs to achieve site-specific integration. For example, encapsidation of the NE-TR75-TTR-hF.IX and NE-TR75-TTR-hF.VIII DNAs in liver-targeted synthetic liposomes will allow repeat dosing and sustained transgene expression from these synthetic AAV vectors. Furthermore, encapsidation of NE-TR75-TTR-hF.IX and NE-TR75-TTR-hF.VIII DNA in exosomes will provide a viable approach for the potential gene therapy for hemophilia in children, because exosome-associated AAV-F.IX vectors have been shown to achieve robust delivery to the liver by evading pre-existing humoral immunity to AAV capsids, thereby leading to a phenotypic correction in hemophilia B mice. Thus, the use of a synthetic NE-AAV DNA vector, comprised in a liposome and devoid of AAV capsid proteins, may prove capable of repeat dosing, without inducing an immune response for the potential gene therapy of liver diseases in children.

What is claimed is:

1. A no-end (NE) recombinant adeno-associated virus (rAAV) genome comprising a heterologous gene flanked by recombinant AAV inverted terminal repeats (ITRs), wherein at least one of the recombinant AAV ITRs is a truncated AAV ITR having between about 35 and 95 nucleotides, wherein the truncated AAV ITR is truncated relative to a full-length AAV ITR of the same serotype.

2. The no-end rAAV genome of claim 1, wherein the truncated AAV ITR has about 55-95 nucleotides.

3. The no-end rAAV genome of claim 1, wherein the truncated AAV ITR has about 35, about 55, about 75, or about 95 nucleotides.

4. The no-end rAAV genome of claim 1, wherein the truncated AAV ITR has about 75 nucleotides.

5. The no-end rAAV genome of claim 1, wherein the truncated AAV ITR has about 35 nucleotides.

6. The no-end rAAV genome of claim 1, wherein the truncated AAV ITR has about 55 nucleotides.

7. The no-end rAAV genome of claim 1, wherein the truncated AAV ITR has about 95 nucleotides.

8. The no-end rAAV genome of claim 1, wherein the no-end rAAV genome does not comprise a nucleotide sequence capable of encoding AAV capsid protein(s).

9. The no-end rAAV genome of claim 1, wherein the truncated AAV ITR further comprises one or more nucleic acid mutations, relative to a full-length AAV ITR of the same serotype.

10. The no-end rAAV genome of claim 9, wherein the nucleic acid mutations are stabilizing mutations and/or result in more efficient ligation than a truncated AAV ITR which does not comprise said mutation(s).

11. The no-end rAAV genome of claim 1, wherein the truncated AAV ITR is truncated relative to a full-length ITR of AAV serotype 1, 2, 3, 4, 5, or 6.

12. The no-end rAAV genome of claim 1, wherein the truncated AAV ITR is truncated relative to a full-length ITR of AAV serotype 2.

13. The no-end rAAV genome of claim 1, wherein the truncated AAV ITR is truncated relative to SEQ ID NO: 2.

14. The no-end rAAV genome of claim 1, wherein the truncated AAV ITR comprises a nucleotide sequence having at least 90%, at least 95%, at least 98%, or at least 99% identity to the nucleotide sequence of any one of SEQ ID NOs: 7-11.

15. The no-end rAAV genome of claim 1, wherein the truncated AAV ITR comprises the nucleotide sequence of any one of SEQ ID NOs: 7-11.

16. The no-end rAAV genome of claim 1, wherein the truncated AAV ITR comprises the nucleotide sequence of SEQ ID NO: 9.

17. The no-end rAAV genome of claim 1, wherein the truncated AAV ITR comprises the nucleotide sequence of SEQ ID NO: 7 or SEQ ID NO: 11.

18. The no-end rAAV genome of claim 1, wherein the truncated AAV ITR comprises the nucleotide sequence of SEQ ID NO: 8.

19. The no-end rAAV genome of claim 1, wherein the truncated AAV ITR comprises the nucleotide sequence of SEQ ID NO: 10.

20. The no-end rAAV genome of claim 1, wherein the heterologous gene is a gene associated with one or more of the following diseases or disorders: Alpha 1-Antitrypsin Deficiency, Phenylketonuria, Wilson Disease, Acute Intermittent Porphyria, Familial Hypercholesterolemia, Crigler-Najjar Syndrome, ATTR Amyloidosis, Methylmalonic Acidemia, a Mucopolysaccharidosis, Glycogen Storage Disease Type Ia, Ornithine Transcarbamylase Deficiency, liver fibrosis, Hemophilia A, Hemophilia B, and Hemophilia C.

21. The no-end rAAV genome of claim 1, wherein the heterologous gene is a gene associated with one or more of the following diseases or disorders: liver fibrosis, Hemophilia A, Hemophilia B, and Hemophilia C.

22. The no-end rAAV genome of claim 1, wherein the heterologous gene encodes albumin, Factor VIII (F.VIII), or Factor IX (F.IX).

23. The no-end rAAV genome of claim 1, further comprising a promoter.

24. The no-end rAAV genome of claim 23, wherein the promoter is a liver-specific promoter and/or a liver cancer cell-specific promoter.

25. The no-end rAAV genome of claim 23, wherein the promoter comprises any one of: human liver cell-specific trans-thyretin (TTR) promoter, human α-fetoprotein promoter (AFP), human α1-antitrypsin (hAAT) promoter, human F.IX promoter, human F.VIII promoter, and liver promoter 1 (LP1).

26. The no-end rAAV genome of claim 1, wherein both recombinant AAV ITRs are truncated, relative to a full-length AAV ITR of the same serotype.

27. A composition comprising the no-end rAAV genome of claim 1.

28. The composition of claim 27, wherein the composition comprises an AAV particle, a liposome, a nanoparticle, or an exosome.

29. The composition of claim 28, wherein the AAV particle is an AAV serotype 1, 2, 3, 4, 5, 6, 7, 8, 9, 10, 11, 12, or 13 particle.

30. The composition of claim 28, wherein the nanoparticle comprises a lipid nanoparticle.

31. The composition of claim 28, wherein the exosome comprises an exosome-associated rAAV.

32. A method of treating a disease or disorder, the method comprising administering the no-end rAAV genome of claim 1 or the composition of claim 27 to a subject in need thereof.

33. The method of claim 32, wherein the subject is a human.

34. The method of claim 32, wherein the subject is a human child.

35. The method of claim 32, wherein the no-end rAAV genome or the composition is administered intramuscularly, intravenously, subcutaneously, intrathecally, intraperitoneally, or by direct injection into an organ or a tissue of the subject.

36. The method of claim 32, wherein the disease or disorder is selected from the group consisting of: Alpha 1-Antitrypsin Deficiency, Phenylketonuria, Wilson Disease, Acute Intermittent Porphyria, Familial Hypercholesterolemia, Crigler-Najjar Syndrome, ATTR Amyloidosis, Methylmalonic Acidemia, a Mucopolysaccharidosis, Glycogen Storage Disease Type Ia, Ornithine Transcarbamylase Deficiency, liver fibrosis, Hemophilia A, Hemophilia B, and Hemophilia C.

37. The method of claim 32, wherein the disease or disorder is selected from the group consisting of: liver fibrosis, Hemophilia A, Hemophilia B, and Hemophilia C.

38. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 9.

39. The recombinant AAV ITR of claim 38, wherein the recombinant AAV ITR comprises the nucleotide sequence of SEQ ID NO: 9.

40. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 7.

41. The recombinant AAV ITR of claim 40, wherein the recombinant AAV ITR comprises the nucleotide sequence of SEQ ID NO: 7.

42. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 8.

43. The recombinant AAV ITR of claim 42, wherein the recombinant AAV ITR comprises the nucleotide sequence of SEQ ID NO: 8.

44. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 10.

45. The recombinant AAV ITR of claim 44, wherein the recombinant AAV ITR comprises the nucleotide sequence of SEQ ID NO: 10.

46. A recombinant adeno-associated virus (AAV) inverted terminal repeat (ITR) comprising a nucleotide sequence having at least 90%, at least 95%, at least 98%, or at least 99% identity to the nucleotide sequence of SEQ ID NO: 11.

47. The recombinant AAV ITR of claim 46, wherein the recombinant AAV ITR comprises the nucleotide sequence of SEQ ID NO: 11.

48. The no-end rAAV genome of any one of claims 1-26 or the composition of any one of claims 27-31, further comprising a nucleotide sequence encoding an AAV Rep protein.

49. The no-end rAAV genome or composition of claim 48, wherein the heterologous gene is integrated into human chromosome 19 (19q13.3).

50. The no-end rAAV genome of claim 1, wherein the heterologous gene comprises a: Serpin Family A Member 1 (SERPINA1) gene, gene encoding Phenylalanine Hydroxylase (PAH), ATPase Copper Transporting Beta (ATPB7) gene, Hydroxymethylbilane Synthase (HMBS) gene, low density lipoprotein receptor (LDLR) gene, UDP Glucuronosyltransferase Family 1 Member A Complex Locus (UGTIA) gene, transthyretin (TTR) gene, methylmalonyl Coenzyme A mutase (MMUT) gene, Mucopolysaccharidosis Type I (MPS I) gene, Mucopolysaccharidosis Type II (MPS II) gene, Mucopolysaccharidosis Type IIIA (MPS IIIA) gene, Mucopolysaccharidosis Type IIIB (MPS IIIB) gene, Mucopolysaccharidosis Type IIIC (MPS IIIC) gene, Mucopolysaccharidosis Type IIID (MPS IIID) gene, Mucopolysaccharidosis Type IVA (MPS IVA) gene, Mucopolysaccharidosis Type IVB (MPS IVB) gene, Mucopolysaccharidosis Type V (MPS V) gene, Mucopolysaccharidosis Type VI (MPS VI) gene, Mucopolysaccharidosis Type VII (MPS VII) gene, Mucopolysaccharidosis Type IX (MPS IX) gene, glucose-6-phosphatase catalytic-subunit (G6PC) gene, Ornithine Transcarbamylase (OTC) gene, gene encoding human serum albumin, gene encoding clotting Factor VIII (F.VIII), or gene encoding clotting Factor IX (F.IX).

51. The no-end rAAV genome of claim 1, wherein the heterologous gene encodes: α-1 anti-trypsin protein, phenylalanine hydroxylase, ATPase Copper Transporting Beta, porphobilinogen deaminase, Hydroxymethylbilane Synthase, low-density lipoprotein receptor, uridine diphosphoglucuronate glucuronosyltransferase, transthyretin protein, methylmalonyl Coenzyme A mutase, α-L-iduronidase, iduronate sulfatase, heparan N-sulfatase, α-N-acetylglucosaminidase, acetyl-CoA α-glucosaminide acetyltransferase, N-acetylglucosamine 6-sulfatase, N-acetylgalactosamine-6-sulfatase, β-galactosidase, α-L-iduronidase, N-acetylgalactosamine 4-sulfatase, β-glucuronidase, hyaluronidase, glucose-6-phosphatase, ornithine Transcarbamylase, human serum albumin, clotting Factor VIII (F.VIII), or clotting Factor IX (F.IX).


SEQUENCE LISTING (11 sequences)

SEQ ID NO: 1 - DNA, 143 bp, Artificial Sequence
  Synthetic
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaa

SEQ ID NO: 2 - DNA, 145 bp, Artificial Sequence
  Synthetic
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 146 bp, Artificial Sequence
  Synthetic
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggc

SEQ ID NO: 4 - DNA, 145 bp, Artificial Sequence
  Synthetic
ttggccactccctctatgcgcgctcgctcactcactcggccctggagaccaaaggtctccagactgccggcctctggccggcagggccgagtgagtgagcgagcgcgcatagagggagtggccaactccatcatctaggtttgcc

SEQ ID NO: 5 - DNA, 167 bp, Artificial Sequence
  Synthetic
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 6 - DNA, 142 bp, Artificial Sequence
  Synthetic
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggta

SEQ ID NO: 7 - DNA, 35 bp, Artificial Sequence
  Synthetic
gggcaaaggcccgggcaaagcccgggcctttgccc

SEQ ID NO: 8 - DNA, 55 bp, Artificial Sequence
  Synthetic
ggaggccgcccgggcaaagcccgggcgtcgggcgacctttggtcgcccggcctcc

SEQ ID NO: 9 - DNA, 75 bp, Artificial Sequence
  Synthetic
gctcgctcactgaggccgcccgggcaaagcccgggcgtcgggcgacctttggtcgcccggcctcagtgagcgagc

SEQ ID NO: 10 - DNA, 95 bp, Artificial Sequence
  Synthetic
ctgcgcgctcgctcgctcactgaggccgcccgggcaaagcccgggcgtcgggcgacctttggtcgcccggcctcagtgagcgagcgagcgcgcag

SEQ ID NO: 11 - DNA, 35 bp, Artificial Sequence
  Synthetic
ggggcaaagcccgggcaaagcccgggctttgcccc